(19) United States
(12) Patent Application Publication
(10) Pub. No.: US 2009/0152374 A1
(43) Pub. Date: Jun. 18, 2009

(54) Gel-Based Liquid Dispensing Device

(75) Inventors: Colin Litten-Brown, Berkshire (GB); Guy Edward Naish, Oxfordshire (GB); Thomas McGee, Cottesloe, WA (US)

Correspondence Address: NORRIS, MCLAUGHLIN & MARCUS, 875 THIRD AVE, 18TH FLOOR, NEW YORK, NY, 10022, US

(73) Assignee: GIVAUDAN SA, Vernier (CH)

(21) Appl. No.: 12/097,852

(22) PCT Filed: Dec. 7, 2006
(86) PCT No.: PCT/CH06/00686

§ 371 Date: Oct. 3, 2008

Related U.S. Application Data
(60) Provisional application No. 60/753,184, filed on Dec. 22, 2005.
(60) Provisional application No. 60/800,910, filed on May 16, 2006.

(51) Int. Cl. A61L 9/012

(52) U.S. Cl. 239/60

(57) ABSTRACT

An active volatile liquid-dispensing device comprising a gel and a separate source of active volatile liquid (“the source”), the gel
(a) comprising volatile material the emission of which causes the reduction in size of least one physical property of the gel, and
(b) being equipped with a screening means that screens the source from the atmosphere, which screening means is caused to open by the size reduction in physical property, thus causing a progressively greater exposure of the source to the atmosphere with passage of time, the exposure of the gel to the atmosphere remaining essentially constant.

The physical property utilised is usually reduction in weight or shrinkage in physical dimensions.

The devices permit a more controlled release of active into the atmosphere and a longer life, and are especially useful as air fresheners.

This invention relates to devices for dispensing active volatile materials into an atmosphere and more particularly to those comprising gels.

Gel-containing devices for dispensing active volatile materials into an atmosphere are common. By “active volatile material” (hereinafter “active”) is meant a volatile material whose presence in an atmosphere is desired for the achievement of some purpose. Such materials include insecticides, fungicides, odour counteractants, and most particularly fragrances. Gel-based air fresheners, which emit fragrance into an atmosphere, are a particularly common example. The working substance is a gel, which may be either aqueous or non-aqueous, in which is incorporated the active, generally dissolved in a carrier solvent. As the active is emitted, the gel shrinks, this shrinkage providing an excellent indication of the approaching end of life of the device and the need for a refill or replacement. The rate of emission depends on ambient conditions of temperature, humidity and air flow. The problem with such systems is that they tend to lose proportionally more volatile material initially and less towards the end of the product life. Such gels will also start as a uniform block of material which may be coloured, transparent, translucent or opaque, but which shrink down at the end to an unsightly, irregular lump. Other types of gel can contain a high loading of fragrance but these often show limited change in physical appearance over the life of the product such that the end point is often unclear.

It has now been found that it is possible to make a gel-based active-dispensing device in which these disadvantages are substantially or completely overcome. The invention therefore provides an active volatile liquid-dispensing device comprising a gel and a separate source of active volatile liquid (“the source”), the gel

(a) comprising volatile material the emission of which causes the reduction in size of least one physical property of the gel, and
(b) being equipped with a screening means that screens the source from the atmosphere, which screening means is caused to open by the size reduction in physical property, thus causing a progressively greater exposure of the source to the atmosphere with passage of time, the exposure of the gel to the atmosphere remaining essentially constant.

The invention additionally comprises a method of maintaining a relatively uniform presence of an active volatile substance in an atmosphere, comprising the evaporation of the volatile substance from a source of the substance, the source being progressively more exposed to the atmosphere by the opening of a screening means that screens the source from the atmosphere and which opening is driven by the reduction in physical property of a gel different from the source, the exposure of the gel to the atmosphere remaining essentially constant.

By “physical property” is meant any physical characteristic of a gel that will change with evaporation of solvent. The normal physical properties in this case are reduction in weight and shrinkage in physical dimensions (length, breadth, height, volume), and most gels will both lose weight and reduce in physical size as solvent evaporates. All of these can be used in the working of this invention, as will be further described hereinunder.

The source of active volatile liquid for use in this invention may be any suitable source. For example, it may be a porous wick of the type well known to the art, which draws active from a reservoir. It may also be a volatile liquid behind a semi-permeable membrane. It may also be a sublimable material of the type known to the art. In a particular embodiment, it may be a gel. This gel is additional to the gel whose change in physical property causes the progressively larger exposure of the source to the atmosphere. Suitable gels are well known to the art and the skilled person can easily select a suitable material for any given application. Examples of typical gel materials known to the art and widely used include waxes, polyamide polymers, hydrocarbon oils containing at least one of diblock, triblock, radial block and multiblock polymers. Such materials are used, for example in candles, and descriptions of such materials can be found in, for example, U.S. Pat. Nos. 3,615,289; 3,645,705; 3,819,342, 4,449,987, 5,578,089, 6,054,417 and 6,111,055 and International Application PCT/US96/13993. Not all such materials are suitable for use in this invention, but using only routine experimentation, the skilled person can easily select a suitable material for any application of this invention.

The gel equipped with the screening means (which will hereinafter be referred to as “the primary gel”, regardless of whether the source is or is not a gel) has the function of progressively exposing more of the source to the atmosphere. To do this, it must be such that a physical property must reduce in size as the gel loses volatile material. As hereinabove mentioned, the screening means will generally be driven either by the physical shrinkage of the gel or its loss of weight. The volatile material in the primary gel is there purely for the purposes of this loss. It is selected from water or blends of water with water-miscible organic solvents, typically ethanol.

An important feature of the invention is that the primary gel itself should not itself become more exposed to the atmosphere as the screening means. Such an exposure would lead to an uneven progressive exposure of the source to the atmosphere and therefore an uneven dissemination of fragrance. It is not critical that the exposure of the primary gel to the atmosphere be kept absolutely constant; the skilled person will realise that small variations in either direction can be tolerated. There are many ways of achieving such exposure that will be evident to the art. Some of these will be described hereinafter in greater detail, but a typical arrangement comprises the primary gel being held in a separate compartment with non-variable ports providing exposure to the atmospher4e and release of material to permit the desired property reduction.

Because the primary gel provides the means that exposes gradually more of the source to the atmosphere, it should selected with the desired physical property in mind. For example, gels based on carrageenan have a significant shrinkage and are particularly useful in this invention. Other types of gel may also be used, and if the fragrance source is also a gel, the gels may be the same or different.

Typical examples of primary gels are the following:

### Clear Carrageenan-Based Gel

| | | % by Weight | |
|---|---|---|---|
| Ingredients | Suppliers | A | B |
| Demin Water | | 68.0 | 61.2 |
| Potassium Chloride | | 0.22 | 0.2 |
| Sodium Dihydroacetate | Lonza Inc | 0.11 | 0.1 |
| Genugel ™RLV (Carrageenan) | CP Kelco | 1.67 | 1.5 |
| Propylene Glycol | | 2.22 | 2.0 |
| Eumulgin ™ B3 | Cognis | 3.33 | 3.0 |
| Cetomacrogol ™ 1000 BP | Croda | 13.33 | 12.0 |
| (Ceteareth-20) | | | |
| Isopar ™M | Exxon Mobil | — | 6.0 |
| | Corporation | | |
| Dowanol ™DPM | Univar | — | 2.0 |
| Dowanol ™ TPM | Univar | — | 2.0 |
| Denatured Ethanol | | 11.12 | 10.0 |
| Total | | 100.00 | 100.0 |

The following gels are typical example of gels useful as a source of active volatile liquid.

### Anhydrous Polymeric Gel

| Ingredients | Suppliers | % by Weight |
|---|---|---|
| Lithene ™ N4-9000 10 MA | Synthomer | 17.14 |
| Fragrance | Givaudan | 80.00 |
| Jeffamine ™ D400 | Hunstman | 2.86 |

### Non-Anhydrous Polymeric Gel

| Ingredients | Suppliers | % by Weight |
|---|---|---|
| 1. Lithene ™ N4-9000-MA10 | Synthomer | 17.00 |
| 2. Fragrance | Givaudan | 75.00 |
| 3. Steol ™ CS-460 | Stepan | 4.00 |
| 5. Jeffamine ™ T-403 | Hunstman | 4.00 |
| | | 100.00 |

| Ingredients | Suppliers | % by Weight |
|---|---|---|
| 1. Lithene N4-9000-MA10 | Synthomer | 18.00 |
| 2. Fragrance (UN059979/00) | Givaudan | 77.00 |
| 3. Steol CS-460 | Stepan | 4.00 |
| 4. Jeffamine T-403 | Hunstman | 1.00 |

### Amide-Terminated Polyamide Gel

| Ingredients | Suppliers | % by Weight |
|---|---|---|
| Sylvaclear ™ gel (A 200) | Arizona Chemical | 52 |
| Fragrance | Givaudan | 48 |

It is to be emphasised that these formulae are only exemplary and the skilled person will be able to make many other gels that fall within the scope of this invention with only routine experimentation.

The primary gel is equipped with a screening means. By “equipped” is meant that there is associated with the primary gel a screening means such that the physical property size reduction of the primary gel causes the screening means progressively to expose more of the fragrance source. This necessitates a direct or indirect physical connection between primary gel and screening means that translates physical property reduction into movement of the screening means. This may be achieved by any convenient means. Some non-limiting examples include the following:
the screening means is a barrier that rests on top of the primary gel and moves downwards as the gel shrinks, the barrier movement gradually exposing more of the active source;
the weight of the primary gel holds closed an openable barrier, against the urging of a force (typically a resilient member, such as a spring) seeking to open the barrier, the weight reduction of the primary gel permitting force-induced movement that allows the openable barrier to open.

The skilled person will readily be able to conceive of a number of possible variants that are not described here, but that lie within the scope of the invention.

The devices according to this invention are easily made from any suitable material, for example, metal, wood, plastics, paper, cardboard and ceramics. Whatever materials are used must be able to be used satisfactorily with any substance with which they come into contact, such as gels, actives and solvents, but this is well within the skill of the art. Preferred materials are plastics, which can be easily and cheaply moulded into desirable and attractive shapes and colours, and which can be made translucent.

The active volatile substance for release into the atmosphere may be any such substance whose presence is desired in the atmosphere and which can be released from the sources hereinabove described. The most popular substances are fragrances, used in air fresheners, but they can also be other substances, for example, odour maskers, insecticides, fungicides and disinfectants.

The devices may be single use, but they are preferably refillable, in that components or all of one or both of the source and the primary gel may be replaced, such that the device may continue to operate. The nature of the replacement will naturally depend on the form and nature of the particular device; for example, a device having a gel as source will require a replacement gel, but a device having a porous wick as source may need only a replacement reservoir of active and the wick can be reused. In each case, the design of such replacement components is within the skill of the art. The device can thus be put back into operation with a minimum of interruption of emission.

The devices according to this invention are unusually effective in maintaining a relatively uniform presence of an active in an atmosphere. They are also very effective at giving an indication of end of life, so that the device may either be replaced or refilled.

The invention is now further described with reference to the accompanying drawings, which depict preferred embodiment and which are intended to be in any limiting of the scope of the invention.

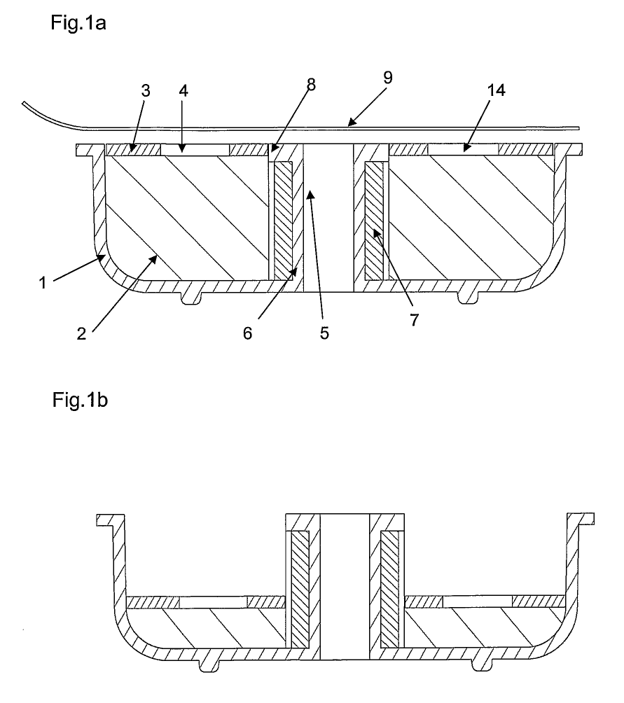

FIGS. 1a and 1b depict vertical cross-sections through an embodiment of the invention in a pre-use and a use situation.

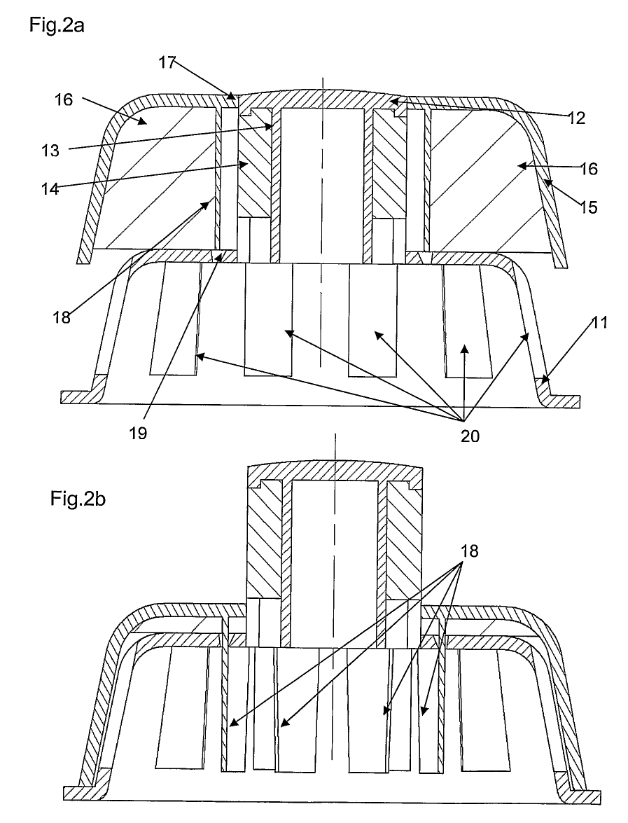

FIGS. 2a and 2b depict vertical cross-sections through a further embodiment of the invention in a pre-use and a use situation.

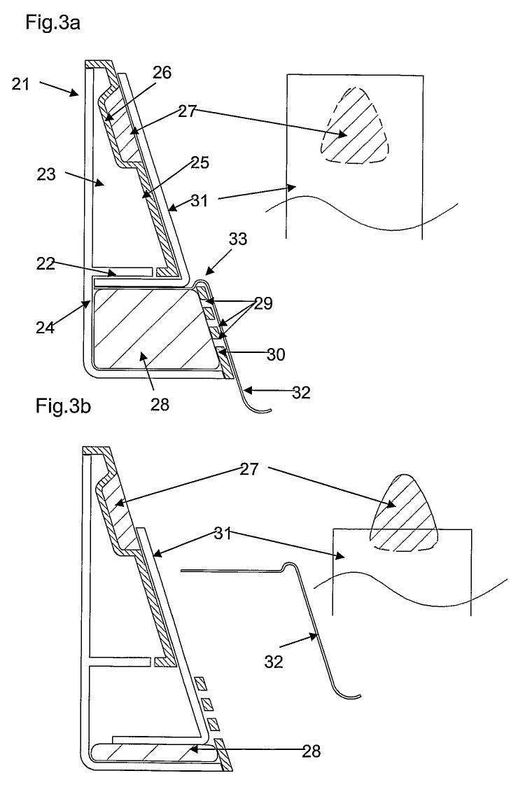

FIGS. 3a and 3b depict vertical cross-sections through a further embodiment of the invention in a pre-use and a use situation.

In FIGS. 1a and 1b, 2a and 2b, and 3a and 3b, the operation of the devices utilises shrinkage in physical size with time. In FIGS. 4a and 4b, the device utilises reduction in weight with time.

In FIGS. 1a-b, 2a-b and 3a-b, the primary gel used has the formula A as hereinabove described, and the active volatile liquid source is the anhydrous polymeric gel hereinabove described.

In FIGS. 1a and 1b, the device has the form of a shallow basin 1 having vertical sides, which is filled with a primary gel 2, this gel being able to shrink substantially through loss of solvent. On top of the primary gel 2 rests a plate 3, this plate moving down the basin as the gel shrinks, the solvent exiting from the gel via a series of ports 4 in the plate. Rising vertically from the centre of the base of the basin is an essentially cylindrical projection 5. Formed circumferentially around the cylindrical surface of this projection is a series of indentations 6 which are filled with a fragrant secondary gel 7. A central port 8 in the plate 3 allows this projection to pass through, and as the gel 2 shrinks, the plate 3 moves downwards (as shown in FIG. 1b).

The operation of the device is started by the removal of a protective foil 9 from the top of the device. Evaporation of the volatile liquid from the primary gel then begins, with consequent shrinkage. As the plate 3 moves downwards as the primary gel 2 shrinks, as shown in FIG. 1b, the downwardly-moving plate exposes more of the secondary gel and allows the fragrance on the exposed secondary gel to evaporate into the atmosphere, while the secondary gel beneath to plate 3 is prevented from evaporating. Thus, a controlled emission of fragrance is achieved. The degree to which fragrance is released can be predetermined by the selection of an appropriate primary gel, something that is well within the skill of the art and requiring only routine experimentation.

In the embodiment of FIGS. 2a-b, a device has a base member 11, in the shape of an inverted bowl. There extends upwardly from the top of the base member a centrally-located cylindrical member 12, which is provided around its circumferences with recesses 13 containing fragrant gel 14. On top of the base member 11 rests a barrier member 15, this barrier having a shape closely matching that of the base member. The barrier member does not rest directly on the base member 11, but on a primary gel 16 that has been provided around the circumference of the barrier member 15. The barrier member 15 has at the top thereof a port 17 through which the cylindrical member 12 fits. In order to maintain the barrier member 15 in register with the base member as the primary gel shrinks, there extends vertically downwards from the inner surface of the top of the barrier member a series of elongate projections 18, which fit into matching slots 19 in the top of the base member 11.

When evaporation of the liquid in the primary gel commences, the liquid escapes through vents 20 in the side of the base member 11. The barrier member then begins to descend, the elongate projections 18 fitting within the corresponding slots 19 and helping keep base member and barrier member aligned. As the barrier member 15 descends, it gradually exposes the recesses 13 with the fragrant gel 14, such that the emission of fragrance is controlled.

In the embodiment of FIGS. 3a-b, a base member 21 has a vertical cross section approximating to a tall right-angled triangle. This base member is split by a horizontal partition 22 into two hollow compartments, an upper compartment 23 and a lower compartment 24. The upper compartment is empty and there is formed on the hypotenuse of the triangle at this upper compartment (the “upper hypotenuse” 25) a recess 26, which is filled with a fragranced gel 27. The lower compartment contains a primary gel 28, and the evaporating liquid from this gel escapes from the lower compartment through vents 29 formed in a lower hypotenuse 30 that bounds the lower compartment.

Evaporation of solvent and fragrance is initially prevented by two cover plates, an upper cover plate 31 and a lower cover plate 32. The upper cover plate 31 has a shape corresponding to the upper hypotenuse 25 and the partition 22. In pre-use condition, it rests on the lower cover plate 32, whose shape corresponds to the partition 22 and the lower hypotenuse 30, thus covering the vents 29. In a pre-use configuration, the upper cover plate 31 is thus separated from contact with the primary gel 28 by the lower cover plate. The smaller drawing to the right of FIG. 1a shows the ghost outline of the fragranced gel 27 in its recess, behind the upper cover plate 31. The lower cover plate 32 has a slight upwards protrusion 33 where the two planes of the cover plate meet, which engages with the upper edge of the lower hypotenuse 30, to provide a positive locking in place, such that the lower cover plate cannot slip out by accident. The plane of the lower hypotenuse 30 extends outwardly from the plane of the upper hypotenuse 25 by the thickness of the upper cover plate, so that the upper cover plate sides inside the lower hypotenuse as it moves downwards.

In operation as shown in FIG. 3b, the lower cover plate 32 is pulled out, This allows the primary gel 28 to commence shrinking by evaporation of solvent through the vents 29 and the upper cover plate 31 to come into direct contact with the primary gel. As the primary gel shrinks, the upper cover plate moves downwards, exposing to an increasing extent as it does so the fragranced gel 27, as shown in the part-ghost outline to the right of FIG. 3b.

1. An active volatile liquid-dispensing device comprising a gel and a separate source of active volatile liquid, the gel
(a) comprising volatile material the emission of which causes the reduction in size of least one physical property of the gel, and
(b) being equipped with a screening means that screens the source from the atmosphere, which screening means is caused to open by the size reduction in physical property, thus causing a progressively greater exposure of the source to the atmosphere with passage of time, the exposure of the gel to the atmosphere remaining essentially constant.

2. A device according to claim 1, in which the physical property whose change drives the screening means is shrinkage in physical dimensions.

3. A device according to claim 1, in which the physical property whose change drives the screening means is reduction in weight.

4. A device according to claim 1, in which the source of active volatile material is a gel other than the gel driving the screening means.

5. A device according to claim 2, in which the screening means is a barrier that rests on top of the gel and moves downwards as the gel shrinks, the barrier movement gradually exposing more of an active source initially covered completely by the barrier.

6. A device according to claim 3, in which the weight of the primary gel holds closed an openable barrier, against the urging of a force seeking to open the barrier, the weight reduction of the primary gel permitting force-induced movement that allows the openable barrier to open.

7. A method of maintaining a relatively uniform presence of an active volatile substance in an atmosphere, comprising evaporation of the volatile substance from a source of the substance, the source being progressively more exposed to the atmosphere by the opening of a screening means that screens the source from the atmosphere and which opening is driven by the reduction in physical property
